(19) United States
(12) Patent Application Publication
(10) Pub. No.: US 2005/0205451 A1
(43) Pub. Date: Sep. 22, 2005

(54) Contact lens packages

(76) Inventors: Susan K. Brown-Skrobot, Jacksonville, FL (US); Michael G. Tokarski, Ponte Vedra Beach, FL (US)

Correspondence Address: PHILIP S. JOHNSON;JOHNSON & JOHNSON, ONE JOHNSON & JOHNSON PLAZA, NEW BRUNSWICK, NJ, 08933-7003, US

(21) Appl. No.: 10/803,606

(22) Filed: Mar. 18, 2004

(51) Int. Cl. A61B 19/02

(52) U.S. Cl. 206/438; 424/427

(57) ABSTRACT

The invention provides a package for storing medical devices that substantially eliminates reaction of a pharmaceutical agent, antimicrobial agent or both with light, which agent is incorporated into the device or contained in a solution in which the device is packaged.

This invention relates to packages for storing contact lenses. In particular, lens packages in which opacifying agents are used are provided.

### BACKGROUND OF THE INVENTION

Contact lenses have been used commercially to improve vision since the 1950s. Conventional lenses typically are made of so-called hard or soft materials. Some soft contact lenses, such as silicone hydrogel contact lenses, have been designed for continuous wear for periods of up to thirty days. It is believed that this extended wear use of the lenses may result in an increase in microbial infections in the user's eye.

To address the issue of microbial infections, antimicrobial agents, such as silver, have been added to contact lenses, as for example in U.S. application Ser. Nos. 10/028,400, 10/029,526 and 60/428,620 all of which are hereby incorporated in their entireties by reference. By “antimicrobial” agent is meant a compound or composition that inhibits the adhesion or growth of microbes, such as bacteria, on the lens or which is capable of killing the microbes. Further, the use of medical devices, such as contact lenses, to deliver pharmaceutical agents to the eye is known. The pharmaceutical or antimicrobial agent may be incorporated into the body of the lens, coated onto the lens surface, or included in the lens packaging solution. This use of antimicrobial and pharmaceutical agents may be disadvantageous in that the agent may react, such as by degradation or a change in color of the agent, over time due to exposure to light.

Conventional contact lens packages are composed of a molded plastic base and a sheet overlying the top of the base. The cover sheet typically is a foil sheet laminated with another material, such as a polyester, which does not permit transmission of light. However, the base is typically translucent, or sufficiently transparent, to permit the passage of light that may react with a pharmaceutical or antimicrobial agents. Thus, it is desirable to provide packages that block light sufficiently so that reaction of the agent with light is prevented. Preferably, the package also will permit sterilization of the lens or device therein by certain wavelengths of light.

### BRIEF DESCRIPTION OF THE DRAWING

FIG. 1 is one embodiment of a base element of a package of the invention.

### DETAILED DESCRIPTION OF THE INVENTION AND ITS PREFERRED EMBODIMENTS

The invention provides a package for storing contact lenses that substantially eliminates reaction with light of a pharmaceutical agent, antimicrobial agent or both, which agent is incorporated in or coated on the lens or contained in a solution in which the lens is packaged. Additionally, preferably the packages of the invention permit sterilization of the lenses using light in either or both the visible and UV wavelength ranges.

In one embodiment the invention provides a package for storing a contact lens comprising, consisting essentially of, or consisting of a base element, wherein the base element comprises, consists essentially of, or consists of an opacifying agent.

Opacifying agents useful in the packages of the invention are agents that block light sufficiently so that the reaction of light with the antimicrobial or pharmaceutical agent is substantially prevented. Preferably, this is achieved while permitting sufficient light to pass through at least a portion of the package so that either or both the package contents may be visually inspected and sterilization of the package contents using ultraviolet light may be carried out. More preferably, the opacifying agent is selected so that, when it is used in conjunction with the base element material, transmission of light in the wavelengths of less than about 550 nm and greater than 650 nm through the package is substantially prevented.

Any opacifying agent that, when incorporated with the base element material, provides the desired light blocking effect may be used. Suitable opacifying agents include, without limitation, aluminum, aluminum hydrate, aluminum silicate, potassium silicate, aluminum mono- di-, and tri- stearate, barium sulphate, bentonite, burnt amber, calcium carbonate, carbon black, D & C Red No. 7, organic pigments including without limitation, soluble dyes absorbed on or combined with an inorganic carrier, diatomaceous earth, iron oxides, magnesium oxide, magnesium silicate, phthalocyanine blue, phthalocyanine green, quinacridone red,.raw and burnt sienna, titanium dioxide, and the like and combinations thereof.

Additionally, the opacifying agent may be a photochromic that darkens the base element in response to exposure to light. Suitable photochromics include, without limitation. spironapthyloxazines, naphthopyrans, benzopyrans, phenantropyrans, spiropyridobenzoxazines, metal-dithizonates, fulgides/fulgimides, spiro(benzindoline), organometal dithizonates and the like and combinations thereof.

The opacifying agent may be incorporated into the material from which the base element is formed, meaning that the agent may be mixed, blended, or otherwise combined with the base element material prior to formation of the base element. Alternatively, the agent may be placed onto either or both the interior and exterior surface of the base element by any convenient method such as by coating, spraying, printing or the like. Preferably, the opacifying agent is incorporated into the material from which the base element is made.

The base element may be made from any material typically used to package contact lenses. Such materials may be polymers, rubbers, or plastics that are compatible with the chemical and physical properties of the lens, the pharmaceutical or antimicrobial agent, and any solution in which the lens may be stored. Examples of suitable base element materials include, without limitation, polypropylene, polyethylene, nylons, olefin co-polymers, acrylics, rubbers, urethanes, polycarbonates, fluorocarbons, and the like and copolymers and blends of the foregoing. Preferably, the base element material is a metallocene catalyzed polymers or co-polymers made of polypropylene or polyethylene and having a melt flow range of about 15 g/10 minutes to about 44 g/10 minutes as determined by ASTM D-1238. Preferred materials include, without limitation, polypropylene, cyclic polyolefins including without limitation, ZEONOR™ 1060R, Exxon ACHIEVE™ 1605, copolymers of polypropylene and polyethylene, blends such as blends of polypropylene with ZEONOR 1060R, and the like and combinations thereof.

For those embodiments in which the opacifying agent is incorporated into the base element material, the opacifying agent may be combined with the material used to form the base element by any convenient method. The precise amount of agent used will depend upon the agent and base element material selected. Typically, about 0.5 to about 10 weight percent, based on the total weight of the base element material, of the opacifying agent will be used.

Once the opacifying agent and the base element material are combined, the base element may be formed by any number of known methods including, without limitation, injection molding, transfer molding, skin packaging, blow molding, co-injection molding, film extrusion, or film co-extrusion. Preferably, the package of the invention includes a base element combined with a cover element to form the package.

More preferably, the base element is configured so that it forms the base of a blister package. In this embodiment, the cover element preferably is a flexible cover 1 0 sheet made from an adhesive laminate of an aluminum foil and a polypropylene film or any other extruded or co-extruded film that can be sealed to the top surface of the flange in order to form a hermetic seal for the ophthalmic device. Examples of suitable blister packages are disclosed in U.S. Pat. Nos. 4,691,820; 5,054,610; 5,337,888; 5,375,698; 5,409,104; 5,467,868; 5,515,964; 5,609,246; 5,695,049; 5,697,495; 5,704,468; 5,711,416; 5,722,536; 5,573,108; 5,823,327; 5,704,468; 5,983,608; 6,029,808; 6,044,966; and 6,401,915 all of which are hereby incorporated in their entireties by reference.

The package of the invention will be useful for storing any number of types of contact lenses. However, the invention may find its greatest utility when used for the storage of soft contact lenses containing a pharmaceutical agent, antimicrobial agent, or both and which lenses are made from silicone elastomers or hydrogels, which include but are not limited to silicone hydrogels, and fluorohydrogels. Exemplary formulations of such soft contact lens are disclosed in U.S. patent application Ser. No. 60/318,536, Ser. No. 09/532,943 and U.S. Pat. Nos. 5,710,302; 5,998,498; 6,087,415; 5,760,100; 25 5,776,999; 5,789,461; 5,849,811; and 5,965,631 along with WO 9421698, EP 406161, and JP 2000016905 all of which are hereby incorporated by reference in their entireties. Examples of the preferred soft contact lenses for use with the package of the invention are those made from formulations of galyfilcon, senefilcon, etafilcon A, genefilcon A, lenefilcon A, polymacon, balafilcon A, lotrafilcon A. The most particularly preferred contact lenses are made from galyfilcon A, senefilcon A, balafilcon A, and lotrafilcon A.

The invention will be clarified by a consideration of the following, non-limiting example.

### EXAMPLE

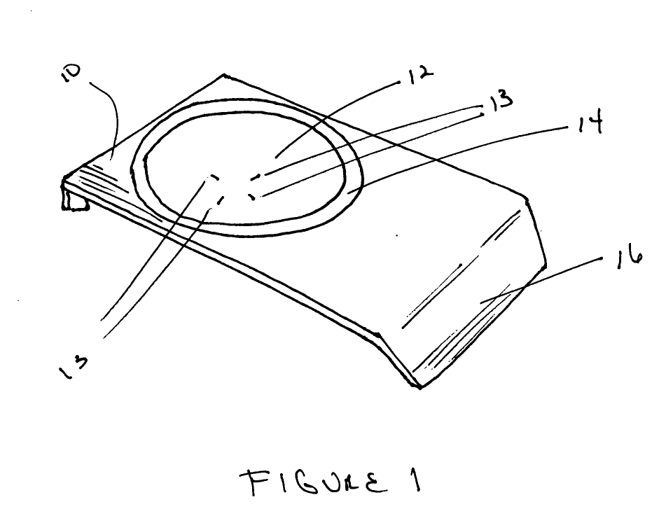

Base elements were made from Exxon ACHIEVE 1605 polypropylene without addition of titanuium dixode. Additional base elements were made by blending titanium dioxide with ACHIEVE 1605 polypropylene to give a final concentrations of titanium dioxide in the material of 4, 2, and 1% by weight. Each of the types of materials was injection molded to form the base elements of contact lens packages. The configuration of the base element was substantially as shown in FIG. 1. The base element had a substantially planar surface 10 and a hemispherical bowl 12 formed therein. Bowl 12 contains a plurality of ribs 13 located off-center of the bowl. A sealing area 14 was located circumferentially about the circular boundary of the first planer surface 10 and bowl 12. A second planar surface 16 extended from the edge of the first planar surface 10 at a non-zero angle in the direction of the bowl.

Contact lenses made from senefilcon A of the formula in Table 1 below were manufactured and stored in jars in deionized water containing 50 ppm of methylcellulose. The lenses were rolled on a jar roller for approximately 18 hours at 19 to 22 degrees C. The lenses were transferred to the base elements and approximately 800 μL of a silver nitrate solution (180 ppm (μg/mL) as silver made by dissolving 0.36 g AgNO₃ in 2000 mL of deionized water in a volumetric flask). After 2½ minutes, the lenses were removed from the solution and rinsed twice in 5 minute increments with DI water. The lenses were added to the individual base elements along with approximately 900 μL of a borate buffered solution (made from approximately 18.524 g boric acid and approximately 3.725 g sodium borate in 2000 mL DI water) containing 50 ppm of methyl cellulose and then the blister pack was heat sealed with a flexible cover element. The lenses were then autoclaved three times in 30 minutes intervals at 122.5 degrees C. and then placed in a 45 degree C. light chamber.

TABLE 1

| | Weight Percent |
|---|---|
| Monomer (80 wt percent) | |
| 2-propenoic acid, 2-methyl-, 2-hydroxy-3[3- | 28.00 |
| [1,3,3,3tetramethyl-1- | |
| [(trimethylsilyl)oxy]disiloxanyl]propxy]propyl | |
| ester | |
| monomethacryloxypropyl terminated mono- | 31.00 |
| n-butyl terminated polydimethylsiloxane | |
| 1000 | |
| N,N-dimethylacrylamide | 24.00 |
| polyvinyl pyrrolidone 360,000 | 7.00 |
| tetraethyleneglycol dimethacrylate | 1.50 |
| 1:1 (wt) blend of 1-hydroxycyclohexyl phenyl | 0.48 |
| ketone and bis(2,6-dimethoxybenzoyl)-2,4-4- | |
| trimethylpentyl phosphine oxide | |
| NORBLOC ™ | 2.00 |
| 2-hydroxyethyl acrylate (“HEMA”) | 6.00 |
| Blue HEMA | 0.02 |
| Diluent (20 wt percent) | |
| 3,7-dimethyl-3-octanol | 100.00 |

The packaged lenses were held in a Caron Photostability Chamber Model 6535 (light intensity approximately 73.497 mW/m²) that emits light in a broad specturm of about 200 to about 800 nm, for a period of 30 days, 60 days, or 180 days. After the set period of time elapsed, the lenses were removed from the packages and tested with a hand held colorimeter (X-Rite XP64). Values in the anges of L-a-b are measured to evaluate shifts in color. The “L” is an indication of how light the color is (positive number=more white and negative number=more black); “a” indicates red-green shifts (positive number=more red and negative numbers=more green); and “b” indicates the yellow-blue shifts (positive number=more yellow and negative numbers=more blue). The established range for L, a, b values is between −100 to +100. Due to the blue visibility tint in the senefilcon contact lenses, shifts in color were mainly seen in the “a” and “b” values.

The legend for the Examples of the tables are as follows:
A—base element without titanium dioxide and with water-based lid stock
B—base element without titanium dioxide and with solvent-based lid stock
C—base element without titanium dioxide and with clear lid stock
D—base element without titanium dioxide and with clear lid stock
E—base element without titanium dioxide and with standard foil lid stock
F—base element without titanium dioxide and with standard foil lid stock wrapped in aluminum foil
G—base element with 4 wt % titanium dioxide and with standard foil lid stock
H—base element with 2 wt % titanium dioxide and with standard foil lid stock

I—base element with 1 wt % titanium dioxide and with standard foil lid stock

Baseline Data

| | A | B | C | D | E | F | G | H | I |
|---|---|---|---|---|---|---|---|---|---|
| Silver | 4.301 | 4.299 | 4.470 | 4.584 | 4.453 | 4.453 | 4.280 | 4.539 | 4.377 |
| (μg) | (0.101) | (0.087) | (0.122) | (0.259) | (0.150) | (0.150) | (0.199) | (0.182) | (0.324) |
| Colorimetry | | | | | | | | | |
| L | N/A | 90.87 | N/A | N/A | 91.02 | N/A | N/A | N/A | N/A |
| | | (0.18) | | | (0.10) | | | | |
| A | | −3.02 | | | −3.25 | | | | |
| | | (0.74) | | | (0.11) | | | | |
| B | | −0.45 | | | −0.47 | | | | |
| | | (0.14) | | | (0.05) | | | | |

One Week Data

| | A | B | C | D | E | F | G | H | I |
|---|---|---|---|---|---|---|---|---|---|
| Silver | 4.219 | 4.384 | 4.200 | 4.201 | 4.313 | 4.256 | 4.283 | 4.586 | 4.288 |
| (μg) | (0.139) | (0.287) | (0.191 | (0.148) | (0.236) | (0.101) | (0.204) | (0.009) | (0.218) |
| Colorimetry | | | | | | | | | |
| L | 91.5 | 91.26 | 91.30 | 91.32 | 91.27 | 91.04 | 91.16 | 91.18 | 91.10 |
| | (0.09) | (0.03) | (0.06) | (0.05) | (0.08) | (0.08) | (0.13) | (0.15) | (0.22) |
| A | −3.04 | −3.14 | −3.02 | −2.92 | −3.17 | −3.24 | −3.27 | −3.05 | −3.21 |
| | (0.15) | (0.05) | (0.08) | (0.08) | (0.06) | (0.0) | (0.1) | (0.03) | (0.19) |
| B | −0.48 | −0.46 | −0.51 | −0.48 | −0.73 | −0.45 | −0.46 | −0.42 | −0.53 |
| | (0.04) | (0.09) | (0.01) | (0.04) | (0.10 | (0.09 | (0.11 | (0.03) | (0.09) |

One Month Data

| | A | B | C | D | E | F | G | H | I |
|---|---|---|---|---|---|---|---|---|---|
| Silver | 3.909 | 4.221 | 4.193 | 3.939 | 43.969 | 4.385 | 4.523 | 4.707 | 4.562 |
| (μg) | (0.146) | (0.105) | (0.107 | (0.159) | (0.105) | (0.104 | (0.125) | (0.208) | (0.110) |
| Colorimetry | | | | | | | | | |
| L | 91.11 | 91.37 | 91.11 | 90.91 | 91.24 | 91.01 | 91.17 | 91.27 | 91.21 |
| | (0.22) | (0.18) | (0.29) | (0.24) | (0.04 | (0.16) | (0.30 | (0.17) | (0.13) |
| A | −2.65 | −2.7 | −2.84 | −2.58 | −2.83 | −3.45 | −3.14 | −3.01 | −3.05 |
| | (0.22) | (0.08) | (0.17) | (0.10) | (0.10) | (0.21) | (0.17) | (0.05 | (0.12) |
| B | −0.19 | −0.21 | −0.38 | −0.13 | −0.4 | −0.43 | −0.55 | −0.47 | −0.57 |
| | (0.14) | (0.10) | (0.21) | (0.08) | (0.08) | (0.22) | (0.12 | (0.06 | (0.07) |

No significant shifts were seen in the baseline versus the one week interval data. At one month, no visible color change in the lenses in the different packages was apparent to the naked eye, but the data indicates that some color changes occurred in packages A through E. The opaque packages, F through I, do not indicate any significant shift in values. Additionally, a decrease in the silver content was found in lenses in packages A through E, while the silver content of packages F through I was substantially unchanged.

1. A package for storing a contact lens, comprising a base element, wherein the base element comprises an opacifying agent.

2. The package of claim 1, wherein the opacifying agent is elected from the group consisting of aluminum, aluminum hydrate, aluminum silicate, potassium silicate, aluminum mono- di-, and tri- stearate, barium sulphate, bentonite, burnt amber, calcium carbonate, carbon black, D & C Red No. 7, organic pigments, soluble dyes absorbed on an inorganic carrier, soluble dyes combined with an inorganic carrier diatomaceous earth, iron oxides, magnesium oxide, magnesium silicate, phthalocyanine blue, phthalocyanine green, quinacridone red, raw sienna, burnt sienna, titanium dioxide, and combinations thereof.

3. The package of claim 1, wherein the opacifying agent is titanium dioxide.

4. The package of claim 1, wherein the opacifying agent is a photochromic compound.

5. The package of claim 4, wherein the photochromic compound is selected from the group consisting of a spironapthyloxazine, naphthopyran, benzopyran, phenantropyran, spiropyridobenzoxazine, metal dithizonate, fulgide, fulgimide, spiro(benzindoline), organometal dithizonate.

6. The package of claim 1, 2, 3, or 4 wherein the opacifying agent is incorporated into a material from which the base element is made.

7. The package of claim 1, 2, 3, or 4 wherein the opacifying agent is coated onto a surface of the base element.

8. The package of claim 1, 2, 3, or 4 wherein the opacifying agent is sprayed onto a surface of the base element.

9. The package of claim 1, 2, 3, or 4 wherein the opacifying agent is printed onto a surface of the base element.

10. The package of claim 1, wherein the base element further comprises a metallocene catalyzed polymer or a metallocene catalyzed co-polymers made of polypropylene.

11. The package of claim 1, wherein the base element further comprises a metallocene catalyzed polymer or a metallocene catalyzed co-polymers made of polyethylene.

12. The package of claim 1 or 11, wherein the package further comprises a flexible cover sheet.

13. The package of claim 12, wherein the cover sheet comprises an adhesive laminate of an aluminum foil and a polypropylene film.

14. The package of claim 12, wherein the package further comprises a contact lens comprising galyfilcon A, senefilcon A, balafilcon A, or lotrafilcon A.

15. The package of claim 14, wherein the contact lens further comprises an antimicrobial agent.

16. The package of claim 14, wherein the contact lens further comprises a pharmaceutical agent.

17. The package of claim 13, wherein the package further comprises a contact lenses comprising galyfilcon A, senefilcon A, balafilcon A, or lotrafilcon A.

18. The package of claim 17, wherein the contact lens further comprises an antimicrobial agent.

19. The package of claim 17, wherein the contact lens further comprises a pharmaceutical agent.
